(19) United States
(12) Patent Application Publication
(10) Pub. No.: US 2004/0223988 A1
(43) Pub. Date: Nov. 11, 2004

(54) Cosmetic composition comprising human serum albumin obtained from transgenic non-human animals

(76) Inventors: Wolfram Eichner, Butzbach (DE); Klaus Sommermeyer, Rosbach (DE)

Correspondence Address: Cathryn Campbell, McDermott Will & Emery, 7th Floor, 4370 La Jolla Village Drive, San Diego, CA, 92122, US

(21) Appl. No.: 10/296,736

(22) PCT Filed: May 28, 2001
(86) PCT No.: PCT/EP01/06058

(30) Foreign Application Priority Data
May 31, 2000 (DE) 10026998.2

(51) Int. Cl. A61K 7/00; A61K 38/17

(52) U.S. Cl. 424/401; 514/12

(57) ABSTRACT

The present invention relates to methods for preparing a cosmetic composition comprising HSA, wherein (a) HSA is obtained from a transgenic non-human animal; and (b) HSA is mixed with a suitable carrier and/or adjuvant. According to a preferred embodiment the invention is directed to a method HSA is obtained from the milk of a lactating bovine. Finally, the invention also relates to the cosmetic composition obtainable according to these methods as well as their use for cosmetic treatment of wrinkles, scars and burn wounds.

The present invention relates to methods of preparing cosmetic compositions comprising human serum albumin (HSA), wherein the HSA is obtained from transgenic animals. The invention is further directed to the cosmetic composition obtainable by this method.

Albumin is the most abundant soluble protein in vertebrates and at the same time represents the protein with the highest concentration in plasma.

In humans, HSA is produced in the liver as a globular, non-glycosylated protein with a molecular weight of 65 kDa. The protein is involved in a large number of essential functions which include regulating blood pressure and osmotic pressure in the circulatory system as well as transporting fatty acids, amino acids, bile pigments and numerous serum molecules.

To maintain normal osmotic pressure in a patient suffering from fluid loss such as in the case of surgical operation, shock, burn or oedema, HSA is administered as a plasma expander. For this purpose, HSA is presently produced by fractionation of blood collected from blood donors. However, this method of preparation inherently comprises the danger of contamination with infectious agents such as hepatitis virus, human immunodeficiency virus, etc. The purification of HSA from human blood therefore comprises the pasteurization of the product and is very expensive.

It is well known that HSA is a major component of human skin. A cosmetic use of HSA isolated from human blood has been proposed but never realized, because such a use would contravene ethical understanding. Due to the expensive method of isolating HSA from blood the cosmetic use of the HSA so obtained is further prohibited by the price of this protein.

Since an increasing number of blood products, such as coagulation factors, are produced by recombinant expression of genes encoding these factors, market dynamics will further increase the relative costs for purification of HSA from blood. In order to ensure sufficient supply for the pharmaceutical use of HSA, various alternative methods for producing HSA have been developed in the art, most of which use recombinant expression of a gene encoding the protein.

Cloning of the cDNA encoding HSA into an expression vector, transformation of bacterial or yeast host cells using this vector, culturing of transformed hosts and isolating the HSA so prepared is disclosed for example in EP 074 646, EP 091 527, EP 366 400 and EP 612 761. One of the problems associated with isolation of HSA from recombinant host cells resides in the fact that residual microbial components, such as bacterial or yeast proteins or lipids, are highly antigenic for humans and the HSA must thus be extensively purified.

The fact that HSA as a carrier protein has an inherent binding activity for numerous microbial products and tissue culture components further complicates the purification scheme and effort.

As an alternative method of preparing recombinant HSA it has been suggested to generate transgenic animals expressing HSA, preferably using expression vectors capable of providing expression in the milk of the transgenic animal. WO91/08216 for example discloses the preparation of an expression vector comprising the complete human genomic HSA gene under the control of 5′ and 3′-regulatory sequences derived from the bovine αS1-casein gene. This vector is used to transform in vitro matured and fertilized oocytes by micro-injection. The oocytes are subsequently cultured in vitro, transferred into cows and allowed to develop into transgenic animals. HSA is secreted into the milk of these transgenic animals.

Further, the HSA cDNA was expressed under the control of the β-lactoglobulin promoter in transgenic animals which also resulted in secretion of HSA into the milk of the animals (WO93/93164).

Methods to isolate recombinant HSA from the milk of transgenic animals have also been disclosed in the art. WO96/02573 for example discloses that HSA can be purified from the milk of transgenic animals by a method, wherein the milk is skimmed, followed by an acid precipitation to remove caseins and chromatography using a cibacron blue-sepharose column, which is suitable to bind specifically HSA and thus allows distinguishing between HSA and the corresponding bovine protein, bovine serum albumin (subsequently designated BSA).

BSA has been widely used as an active compound in cosmetic preparations, such as creams and lotions, to achieve skin conditioning (see CTFA, International Cosmetic Ingredient Dictionary). Kligman and Christopher (J. Soc. Cosmetics Chemists, 16 (1965), p.557-562) in this context disclose that purified solutions of BSA promptly effaces the finer wrinkles of aged facial skin. In a clinical study it was shown that this effect is primarily mechanical and achieved by tightening of the skin when the protein film dries (Kligman, A. M. and Papa, C. M., Journ. Soc. Cosm. Chem., vol. 16 (1965), p. 557). Benhaim and Brun (Parfümerie und Kosmetik, Vol. 770 (1996), p.176-180) even conclude that when it comes to tightening the skin, no active ingredient has up to now been able to achieve an equal performance as BSA, which was also designated the “reference product” in cosmetics.

BSA sofar used in cosmetic preparations was obtained from cow blood at slaughteries.

Besides for the advantageous activity, BSA could be used in cosmetic preparations for several reasons. First of all, humans are well used to contact with products obtained from cows, i.e. proteins, carbohydrates, lipids, fatty acids, etc.; these products in general thus have a low antigenicity for humans. Further, topical application of a protein raises less allergic problems than other modes of application, for example injection. Therefore the cosmetic use of BSA did not require a highly purified protein. BSA was thus available at a price, which allowed incorporation into a cosmetic product.

However, recent reports on transmissible spongiforme encephalopathy diseases (TSE/BSE, bovine spongiforme encephalopathy) and the fact that transmission of these diseases to humans via a cosmetic product could not entirely be excluded resulted in a situation, wherein bovine products had to be removed from cosmetic preparations.

The use of alternative sources for albumin has been disclosed in the prior art. U.S. Pat. No. 4,863,733 for example relates to a method for cosmetic treatment of humans, wherein blood is obtained from a human, albumin is isolated and re-injected into the patient, to achieve skin conditioning in the proximity of scars or implantation areas. While this method may be applied for autologous HSA donations, heterologous donations would again be contrary to ethical principles, comprise the risk of transmitting infections and be too expensive.

Eggs and swine ovaries or placenta have further been proposed as alternative sources for albumin (U.S. Pat. Nos. 2,043,657 and 3,041,245).

EP 180 968 and EP 244 849 both disclose cosmetic preparations containing HSA. It is stated that the HSA may be prepared by recombinant expression in bacteria or yeast cells. However, as outlined above, expression in micro-organisms necessarily leads to contamination with microbial and cell culture antigens. HSA obtained from these sources therefore has to be purified to an extremely high level to obtain a composition which can be used on humans.

The purification would be so expensive that a respective method will not yield a marketable product.

The problem underlying the present invention thus resides in preparing a cosmetic preparation at a marketable price, which comprises an active compound having a superior performance over BSA.

This problem is solved by a method for preparing a cosmetic composition comprising HSA, wherein

(a) HSA is obtained from transgenic non-human animals; and

(b) HSA is mixed with a suitable carrier and/or adjuvant.

The present invention also relates to the cosmetic composition obtainable according to the above method.

The present invention surprisingly discloses that HSA obtained from transgenic non-human animals can be used to prepare cosmetic compositions. Transgenic animals are usually kept in a closed herd management under conditions comparable to good manufacturing practise. Therefore, collection of serum albumin from transgenic animals which were specifically selected, are known to be free of pathogens and kept in isolation from other animals, does not comprise the risk of transmitting infectious diseases, such as BSE/TSE.

According to the present invention, HSA may be obtained from any transgenic non-human animal which expresses the HSA gene. However, HSA is preferably obtained from a bovine, ovine, porcine, equine, rodents or caprine.

For the purposes of the present application the term “HSA” is used to refer to human proteins of the albumin super-family, as originally found in human blood as well as natural or synthetically modified variants thereof. A number of polymorphisms and mutants of human albumin are known to the person skilled in the art (T. Peters, All about Albumin: Biochemistry,. Genetics and Medical Applications, Academic Press Inc., 1996) and are covered by the term “HSA” just as well as fragments of the human protein, comprising at least ⅓ and preferably ⅔ of the protein sequence.

Other variants can be obtained by substituting, inserting or adding nucleotides to the gene encoding HSA and are covered by the term “HSA” as used in the present application as long as the HSA nucleotide sequence so obtained still has a homology of at least 75% with the natural sequence, wherein a homology of at least 85% is preferred and a homology of at least 90% is most preferred.

Methods to transform single cells of non-human animals with heterologous DNA encoding a foreign protein of interest and regulatory sequences for expressing that protein in the transgenic animal, as well as methods of regenerating transgenic animals are well known to the person skilled in the art (WO91/08216; Bondioli et al., Biotechnology, vol. 16 (1991), 265; Ebert et al., Bio/Technology, vol. 9 (1991), 835; Hammer et al., Nature, vol. 315 (1985), 680; Houdebine L. M. (ed), Transgenic Animals—Generation and Use, Harwood Academic Publishers GmbH (1996), Amsterdam; Pinkert C. A. (ed), Transgenic Animal Technology: A Laboratory Handbook. Academic Press, San Diego (1994), Calif.)).

The cells may be transformed with the nucleic acid by any of the numerous methods known in the prior art. For example, transgenic non-human animals may be obtained using a method comprising

(a) introducing the nucleic acid encoding HSA into a suitable non-human recipient cell; and

(b) regenerating a transgenic non-human animal from the recipient cell.

The recipient cell is preferably an embryonic cell but other cell types may also be used. Regeneration of the transgenic non-human animal from the embryonic recipient cell may comprise transfering the cell into a female non-human animal and allowing the embryo to grow therein.

The method for producing transgenic non-human animals may further comprise the cloning of animals. Methods for cloning animals are well known to those skilled in the art (Baguisi et al., Nature Biotech., vol. 17 (1999), 456-461; Campbell et al., Nature, vol. 380 (1996), 64-66; Cibelli et al., Science, vol. 280 (1998), 1256; Kato et al., Science vol. 282 (1998), 2095-2098; Schnieke et al., Science, vol. 278 (1997), 2130-2133; Vignon et al., C. R. Acad. Sci. Paris, Sciences de la vie/Life Sciences vol. 321 (1998), 735-745; Wakayama et al., Nature, vol. 394 (1998), 369-374; Wells et al., Biol. Reprod. vol. 57 (1997), 385-393; Wilmut et al., Nature, vol. 385 (1997), 813) and may readily be applied in accordance with the present invention to prepare a large number of transgenic animals.

In one embodiment HSA is obtained from the milk or blood of the transgenic non-human animal, preferably from the milk of a lactating bovine.

In an alternative embodiment HSA is obtained from an egg of a transgenic bird. The transgenic bird is preferably a chicken. Methods of expressing proteins in transgenic hens so that the protein is transported into the eggs of those hens are known in the art (see for example Morrison et al., Immunotechnology, vol. 4 (1998), p. 115 to 125).

Parts or products of the transgenic animal comprising the HSA, for example the milk or egg, may be directly formulated into a cosmetic preparation. Alternatively, the HSA may be partially or fully isolated therefrom. The present invention thus also provides a method for preparing a cosmetic composition, which comprises the step of isolating HSA from the transgenic animal.

Numerous methods for purifying proteins are known to the person skilled in the art and can be used according to the present invention to obtain purified HSA. If for example HSA is to be isolated from the milk of a transgenic non-human animal, the method of isolation may comprise a clarification step, which is preferably performed by filtration.

Alternatively or in addition to the clarification, the method of isolating HSA may further comprise one or several steps, wherein HSA is precipitated from a solution comprising HSA. HSA may for example be obtained in high purity from the milk or blood of a transgenic non-human mammal by a single precipitation step. Suitable agents capable of precipitating HSA are known in the art and may be identified by the skilled person using simple experiments. Subsequently, HSA may be resuspended in a desired solvent using well known methods. Preferably, the solvent has characteristics which simplify the cosmetic use of HSA (pH, selection of ions).

The method of isolating HSA may further comprise a chromatography purification step, which may be a performed according to any of the large number of chromatography methods known in the art. The use of a affinity- or ion exchange chromatography is preferred.

According to the present invention HSA obtained from transgenic non-human animals need not necessarily be purified to a high degree. The HSA preparation used for formulation of the cosmetic composition may thus for example still comprise a residual amount of BSA in the range of 0-10% by weight of the isolated HSA, preferably in the range of 0.05-2.5%, most preferred in the range of 0.5-1.0% by weight of the isolated HSA.

The cosmetic compounds may further comprise other substances of transgenic animals, such as other proteins, lipids, fatty acids, carbohydrates, etc. As most humans are well used to contact with products from these animals, the risk of allergic reaction upon application of the preparation of the present invention is low.

The cosmetic composition prepared according to a method of the present invention may comprise HSA in any amount suitable for cosmetic formulation. Usually, the amount of HSA will be within the range of 0.1 to 30% and preferably in the range of 1 to 15% by weight of the cosmetic composition. A concentration of HSA in the range of 3 to 8 by weight of the cosmetic composition is most preferred.

A wide variety of carriers and adjuvants for formulation of cosmetic preparations are known to the person skilled in the art (only by way of example it is referred to Jellinek, Kosmetologie, Dr. Alfred Hüthig Verlag; Janistyn, Taschenbuch der modernen Parümerie und Kosmetik, Wissenschaftliche Verlagsgesellschaft Stuttgart; and Bauer et al., Pharmazeutische Technologie, Thieme Verlag). The type(s) of carrier and/or adjuvants to be used for preparation of the cosmetic composition in accordance with the present invention, will therefore depend on the type of cosmetic product to be prepared. Any of the carriers and adjuvants known in the art which are suitable for administration of HSA can be used in a method for preparing a cosmetic composition according to the present invention.

Numerous examples of (oil in water and/or water in oil) cremes, lotions, oils, gels, hydrogels and sun blocking, after-sun as well as after-shave preparations are disclosed in W. Umbach, Kosmetik: Entwicklung, Herstellung u. Anwendung kosmet. Mittel, Thieme, 1995. HSA can be incorporated into any of these preparations by methods well known to the person skilled in the art.

Due to its smoothening and moisturing activity HSA is preferably incorporated into “leave-on” products, such as hydrogels, cremes, sun blocking gels, after-sun and after-shave preparations as well as lippsticks. In accordance with the present invnetion, incorporation of HSA into preparations on the basis of an oil in water or water in oil emulsion and into film forming preparations is especially preferred.

Besides HSA, the cosmetic preparation may comprise one or a number of further active compounds, for example antibacterial or antimycotic compounds.

In a further embodiment, the present invention is directed to a cosmetic composition obtainable according to the methods described in detail above. The cosmetic composition may have any form of known cosmetic compositions but will preferably be formulated as a lotion, a cream, a gel or an oil.

Finally, the present invention also relates to the use of these compositions for skin conditioning in general and specifically to the cosmetic treatment of wrinkles, scars and burn wounds.

1. Method for preparing a cosmetic composition comprising HSA, wherein
(a) HSA is obtained from a transgenic non-human animal; and
(b) HSA is mixed with a suitable carrier and/or adjuvant.

2. Method according to claim 1, wherein HSA is obtained from a bovine, ovine, porcine, equine, rodents or caprine.

3. Method according to claims 1 or 2, wherein HSA is obtained from the milk or blood of the transgenic non-human animal.

4. Method according to one of claims 1 to 3, wherein HSA is obtained from the milk of a lactating bovine.

5. Method according to claim 1 or 2, wherein HSA is obtained from an egg of a transgenic bird.

6. Method according to one of the preceding claims, wherein the step of obtaining HSA comprises a clarification step.

7. Method according to claim 6, wherein the clarification is performed by filtration.

8. Method according to one of claims 1 to 7, wherein the step of obtaining HSA comprises a precipitation of the HSA from a solution containing HSA.

9. Method according to one of claims 1 to 8, wherein the step of obtaining HSA further comprises a chromatography purification step.

10. Method according to claim 9, wherein the chromatography step is performed by affinity- or ion exchange chromatography.

11. Method according to one of claims 1 to 10, wherein the HSA isolated from the transgenic non-human animals comprises a residual amount of BSA in the range of 0-10% by weight of the isolated HSA.

12. Method according to claim 11, wherein the residual amount of BSA is in the range of 0.05-2, 5% by weight of the isolated HSA.

13. Method according to claim 12, wherein the residual amount of BSA in the range of 0.5-1.0% by weight of the isolated HSA.

14. Method according to one of claims 1 to 13, wherein HSA is incorporated into the cosmetic composition in a concentration in the range of 0.1 to 30% by weight of the cosmetic composition.

15. Method according to claim 14, wherein HSA is incorporated into the cosmetic composition in a concentration in the range of 1 to 15% by weight of the cosmetic composition.

16. Method according to claim 15, wherein HSA is incorporated into the cosmetic composition in a concentration in the range of 3 to 8 by weight of the cosmetic composition.

17. Cosmetic composition obtainable according to a method of anyone of claims 1 to 16.

18. Cosmetic composition according to claim 17, which is a lotion, a cream, a gel or an oil.

19. Use of a composition according to one of claims 17 or 18 for cosmetic treatment of wrinkles, scars and burn wounds.
